(19) United States
(12) Patent Application Publication
(10) Pub. No.: US 2010/0174119 A1
(43) Pub. Date: Jul. 8, 2010

(54) PROCESS FOR THE PREPARATION OF ALDEHYDES

(76) Inventors: Laurent Ducry, Sierre (CH); Dominique Roberge, Sierre (CH); Meinrad Mathier, Salgesch (CH); Michael Gottsponer, Visperterminen (CH)

Correspondence Address: HOFFMANN & BARON, LLP, 6900 JERICHO TURNPIKE, SYOSSET, NY, 11791, US

(21) Appl. No.: 12/666,576

(22) PCT Filed: Jun. 30, 2008
(86) PCT No.: PCT/EP08/05309

§ 371 Date: Dec. 23, 2009

(30) Foreign Application Priority Data
Jun. 29, 2007 (EP) 07012799.8

(51) Int. Cl. C07C 45/61

(52) U.S. Cl. 568/420

(57) ABSTRACT

The present invention is related to a method for the production of an aldehyde by reducing an ester of a carboxylic acid with H-DIBAL (diisobutylaluminium hydride).

The present invention is related to a method for the preparation of an aldehyde by reducing an ester of a carboxylic acid with H-DIBAL (diisobutylaluminium hydride).

DE-A1-100 14 298 discloses the reduction of organic compounds with hydrides in a micro-reactor in general. The only example in DE-A1-100 14 298 discloses the reduction of an ester of a carboxylic acid with H-DIBAL at room temperature to get the corresponding alcohol.

WO-A 01/70649 claims the reduction of organic compounds with hydrides in a microreactor and discloses the reduction of an ester of a carboxylic acid to the corresponding alcohol in a microreactor in the presence of DIBAL-H.

WO-A 99/22857 discloses hydrogenation in the presence of hydrogen gas and fluorination reactions in the presence of fluorine gas in a microreactor. The fluorination reactions take place at elevated temperatures of about 20 to 180° C.

EP-A 0855395 discloses DIBAL-H reductions of methyl a ester in laboratory scale at temperatures below −80° C.

Known ester DIBAL-H reductions in textbooks like J. March, Advanced Org. Chem., 3^{rd} Edition (1985), page 397, emphasize very low reaction temperatures of −70° C.

The reduction of organic compounds is widely used in preparative organic chemistry. In addition the preparation of aldehydes from esters is a constant aim in industry. However, aldehydes are very sensitive towards further reduction to the corresponding alcohols. Therefore, direct reduction of alkyl esters often leads to selectivity problems on an industrial scale. Thus, a large number of interesting aldehydes are not available by direct reduction. A further difficulty is the requirement of selective reduction of compounds having more than one ester group wherein only one ester group should be reduced.

To reduce safety problems while handling hydrides on an industrial scale amounts is a constant aim in industry. H-DIBAL is highly flammable and sensitive to decomposition in the presence of water traces. The required achievement and maintenance of protective-gas conditions on an industrial-scale reduction plant is very expensive.

The object of the present invention is therefore to provide a method for the direct reduction of organic esters to aldehydes while avoiding the above mentioned problems. The inventive method should provide a high selectivity for the production of the aldehyde as compared to the formation of the corresponding alcohol. A further object was to provide a method which allows selective reduction of an ester group in the presence of other ester groups which are not intended for reduction. Another object was to reduce the achievement and maintenance of protective-gas conditions on an industrial-scale reduction plant. Furthermore, an object was to provide increased safety for humans, facilities and environment.

These objects are achieved by the method of claim 1.

Provided is a method for the preparation of an aldehyde by reduction of an alkyl ester of a carboxylic acid, said acid being selected from the group consisting of aliphatic, alicyclic, aromatic, heterocyclic and heteroaromatic carboxylic acids, with H-DIBAL (diisobutylaluminium hydride), wherein the ester/H-DIBAL molar ratio is in the range of about 1:1 to about 1:1.2, and wherein the reaction is carried out in a microreactor, and wherein the temperature in the microreactor is controlled in the range of 0 to −50° C., preferably in the range of −18 to −40° C., more preferably in the range of −20 to −40° C.

The above mentioned ester/H-DIBAL molar ratio refers to each ester group of a molecule which can be reduced into an aldehydic group under the conditions given above. It has been found that by reduction of an alkyl ester in a microreactor with H-DIBAL at an ester/H-DIBAL molar ratio (Feed-1/Feed-2) in the range of about 1:1 to about 1:1.2 the corresponding aldehyde can be obtained in remarkably higher amount than the corresponding alcohol. In a further preferred embodiment the molar ester/H-DIBAL ratio is in the range of about 1:1 to about 1:1.1.

Above about 0° C. the aldehyde/alcohol product ratio tends to even out and furthermore the conversion lowers dramatically. Temperatures of about −70 to −80° C. or even below are difficult to achieve in large scale and therefore not suitable from an industrial point of view.

Esters comprising residues formally derived from aliphatic, alicyclic, aromatic, heterocyclic and heteroaromatic carboxylic acids (acid moiety) are therefore aliphatic, alicyclic, aromatic, heterocyclic and heteroaromatic acid esters or carboxylates.

Here and hereinbelow the term “aliphatic acid ester” or “alkanoate” represents an ester comprising at least one linear or branched alkanoic moiety. Said alkanoic moiety optionally comprise further carbocyclic or heterocyclic moieties, further optionally comprising one or more C₁₋₆-alkyl or C₁₋₆-alkoxy substituents, one or more carbon-carbon double or triple bonds, one or more optionally protected hydroxy, amino or keto groups and/or one or more halogen atoms. In a preferred embodiment said alkanoic moiety is a C₃₋₃₀-alkanoic moiety having 3 to 30 carbon atoms.

Here and hereinbelow the term “C₁₋₆-alkyl” represents a linear or branched alkyl group having 1 to 6 carbon atoms, represents for example methyl, ethyl, propyl, isopropyl, butyl, isobutyl, sec-butyl, tert-butyl, pentyl and hexyl.

Here and hereinbelow the term “C₁₋₆-alkoxy” represents a linear or branched alkoxy group having 1 to 6 carbon atoms, for example methoxy, ethoxy, propoxy, isopropoxy, butoxy, isobutoxy, sec-butoxy, tert-butoxy, pentyloxy and hexyloxy.

Preferred examples for the inventive reduction of higher substituted alkyl C₃₋₃₀-alkanoates are optionally N-protected alkyl α-amino acid esters, alkyl acrylates, alkyl malonates, alkyl succinates, alkyl maleinates, alkyl adipates, alkyl malates and alkyl tartrates or derivatives. Derivatives of alkyl tartrates are for example ketals of alkyl malates or acetals of alkyl tartrates as well as other acetal and/or ketal protected hydroxy groups of alkyl C₃₋₃₀-alkanoates. Especially preferred are n-alkyl alkanoates.

According to the present method (4R,5R)-2,2-Dimethyl-1,3-dioxolan-4,5-dicarbaldehyde can be obtained from dimethyl (4R,5R)-2,2-dimethyl-1,3-dioxolan-4,5-dicarboxylate. The product is a starting compound for cyclic urea diols, such as [4R-(4α,5α,6β,7β)]-hexahydro-5,6-di-hydroxy-1,3-bis[(4-hydroxymethyl)phenyl]methyl]-4,7-bis(phenylmethyl)-2H-1,3-diazepin-2-one [CAS 151867-81-1], as HIV protease inhibitors as disclosed in Lam, P. Y. et al, Science 1994, 263, 380-384.

Examples for aliphatic ester with carbocyclic or heterocyclic moieties are methyl phenylacetate, methyl 5-[5-(1-hydroxyheptyl)-tetrahydrofuran-2-yl]-pentanoate and methyl jasmonate.

The term “alkyl ester” in the meaning of the present invention independently means one of the aliphatic, alicyclic, aromatic, heterocyclic and heteroaromatic esters as defined above wherein the alkyl moiety of the alkyl ester group of the respective molecule is an optionally branched alkyl group, optionally said alkyl group comprising one or more halogen atoms, preferably fluorine and/or chlorine. Examples are methyl, ethyl, n-propyl, isopropyl, n-butyl, isobutyl, sec-butyl, tert-butyl, pentyl or hexyl esters.

Accordingly, each “n-alkyl ester” in the meaning of the present invention independently means one of the aliphatic, alicyclic, aromatic, heterocyclic and heteroaromatic esters as defined above wherein the alkyl moiety of the respective ester group of the respective molecule is an n-alkyl group such as methyl, ethyl, n-propyl or n-butyl.

Also accordingly, each “branched alkyl ester” in the meaning of the present invention independently means one of the aliphatic, alicyclic, aromatic, heterocyclic and heteroaromatic esters as defined above wherein the alkyl moiety of the respective molecule is a branched alkyl group such as isopropyl, isobutyl, sec-butyl or tert-butyl.

Here and hereinbelow the term “alicyclic acid ester” represents an ester comprising a cyclic alkanoate moiety, optionally comprising one or more C₁₋₆-alkyl or C₁₋₆-alkoxy substituents, one or more carbon-carbon double or triple bonds, one or more optionally protected hydroxy, amino or keto groups and/or one or more halogen atoms. Alkyl cyclohexanoates are examples thereof.

Here and hereinbelow the term “aromatic acid ester” represents an ester comprising a mono or polycyclic aromatic acid moiety, optionally comprising one or more C₁₋₆-alkyl or C₁₋₆-alkoxy substituents, one or more carbon-carbon double or triple bonds, one or more optionally protected hydroxy, amino or keto groups and/or one or more halogen atoms. Preferred examples are alkyl benzoates, alkyl salicylates, alkyl anisates, alkyl terephthalates, alkyl naphthoates and di n-alkyl naphthodicarboxyllates. Particularly preferred are the respective n-alkyl aromatic acid esters.

Here and hereinbelow the term “heterocyclic acid ester” represents an ester comprising a a mono or polycyclic heterocyclic moiety having at least one ester group attached to the core, optionally having one or more additional substituents selected from the group consisting of C₁₋₆-alkyl, C₁₋₆-alkoxy substituents, one or more carbon-carbon double or triple bonds, one or more optionally protected hydroxy, amino or keto groups and/or one or more halogen atoms. Preferred examples are alkyl pyrrolidine-2-carboxylates (alkyl hygrinates), 1-methylpyrrole-dine-2-carboxylates, alkyl picolinates and alkyl piperinates.

Here and hereinbelow the term “alkyl heteroaromatic acid ester” represents a mono or polycyclic heteroaromatic moiety having at least one ester group attached to the core, optionally having one or more additional substituents selected from the group consisting of C₁₋₆-alkyl, C₁₋₆-alkoxy, one or more optionally protected hydroxy, amino or keto groups and/or one or more halogen atoms. Preferred examples are alkyl nicotinates. For example 6-methylpyridine-3-carbaldehyde is an expensive starting material for the preparation of 5-chloro-6′-methyl-3-[4-(methylsulfonyl)phenyl]-2,3′-bipyridine, the COX-2 inhibitor Etoricoxib (Arcoxia®) and is currently produced in a two step synthesis from cheap synthon methyl 6-methyl-nicotinate. Alkyl furanoic-2-carboxylates are examples thereof.

It has been found that esters comprising such a branched alkyl moiety, i.e. isopropyl, isobutyl, sec-butyl and tert-butyl esters tend not to react with H-DIBAL at temperatures of about −18° C. or lower. Thus, in an alternative method wherein the temperature of the microreactor is controlled to −18° C. or lower the compound subject to H-DIBAL reduction can be a mixed ester, having at least one n-alkyl ester group and at least one branched alkyl ester group in the same molecule. At or below −18° C. said at least one n-alkyl ester group(s), in the presence of said at least one branched ester group(s), predominantly is/are reduced into the corresponding aldehydic group(s) while said branched ester group(s) remain essentially unaffected. In mixed esters particular preferred the n-alkyl ester is a methyl ester group. Furthermore preferred in mixed esters the branched ester group is a tert-butyl ester group.

An example for a mixed ester and the selective reduction is the preparation of tert-butyl (4R,6S)-(6-formyl-2,2-dimethyl-1,3-dioxan-4-yl)acetate [CAS 124752-23-4] which is a common intermediate for various statins. According to the present invention it can be obtained from tert-butyl (4R,6S)-(6-methylcarboxylate-2,2-dimethyl-1,3-dioxan-4-yl)acetate.

It has also been found that the aldehyde/alcohol product ratio increases when the reaction time shortens. Thus, in a further preferred embodiment the reaction time is below 1 min, preferably below 30 seconds.

A “microreactor” shall be defined as a reactor which reaction volumes have dimensions perpendicular to the flow direction of about 10000 micrometers and less.

Preferably the method is carried out by mixing at least two fluids, one of the at least two fluids comprising the ester of an aliphatic, aromatic and heteroaromatic acid (1^{st} reactant), and another fluid comprising a H-DIBAL (diisobutylaluminium hydride) (2^{nd} reactant), and optionally further fluids, said mixing taking place in a microreactor (6) comprising at least one flow path (1) for one of the at least two fluids (A) comprising either the 1^{st} or 2^{nd} reactant, said flow path(s) comprising at least two reaction regions (2), each reaction region comprising an injection point (3) for feeding the other one of the two fluids (B) comprising either the 2^{nd} or 1^{st} reactant, a mixing zone (4) in which the at least two fluids contact each other and a reaction zone (5), and wherein the microreactor optionally provides one or more additional residence time volumes, and wherein in said method one of the at least two fluids comprising either the 1^{st} or 2^{nd} reactant establishes a first flow and wherein at other one of the at least two fluids comprising either the 2^{nd} or 1^{st} reactant is injected into said first flow at least at two injection points (3) along said flow path(s) (1) in a way such that at each injection point only a fraction of the amount necessary to reach completion of the reaction is injected.

The expression “necessary to reach completion of the reaction” means the amount which would have to be added to reach “theoretical” completion of the reaction, for example in a single vessel. In a simple 1:1 reaction stoichiometry this would be equimolar amounts. For a 1^{st} reactant like dimethyl terephthalate or dimethyl adipinate two molar equivalents of H-DIBAL are necessary to complete the reaction.

FIG. 1 and FIG. 2 show two examples of feeding a flow B at various injection points to a flow A. The microreactor (6) in FIG. 1 comprises one flow path with three injection points, the microreactor (6) in FIG. 2 comprises two flow paths each having three injection points. There may be more than two flow paths present, as well as more than three injection points in each flow path. Thus, the 2^{nd} reactant may be fed at the injections points to a first flow generated by the fluid comprising the 1^{st} reactant. From an economical point of view the more expensive and/or more reactive reactant is advantageously fed to the first flow comprising the cheaper and/or less reactive reactant. H-DIBAL is the more reactive reactant but will be in most cases cheaper than the compound to be reduced. To avoid over-reaction it is preferred to feed the compounds to be reduced continuously as Feed-1.

Furthermore, there are no structural limits regarding the injection points, the mixing zones and/or the reaction zones. Only for the reason of better understanding of the parts of the microreactor used in the present invention the microreactors in FIG. 1 and FIG. 2 are depicted as a linear strung-out hollow space. Nevertheless, the flow path(s) (1) may be tortuously bent as known in the art. Furthermore, different mixing zones and/or reaction zones don't need to have the same dimensions in width or length. It is further not necessary to use a microreactor which contains all of the features mentioned above in one physical entity. It is also possible to externally connect additional injection points, mixing zones, reaction zones, each optionally cooled or heated, to a flow path externally.

Feeding only a fraction of the amount necessary to reach completion of the ester reduction while using more than one injection point leads to an increase of the number of hot spots in the microreactor while the temperature rise in each hot spot is reduced as compared to a typical microreactor with only one mixing and reaction zone. In addition, since one of the two compounds is diluted in the first flow comprising the other compound, formation of side products is reduced and yields are increased. Thus, the inventive method provides an improved control over reactions.

In the present invention each of the at least two fluids independently can be a liquid, a gas or a supercritical fluid. Depending on the mixing properties of the mixing zone it is not necessary that the at least two fluids are completely miscible.

In addition to the at least one general flow path, at least one injection point, at least one mixing zone and at least one reaction zone a suitable microreactor for the inventive method may comprise additional structural elements such as temperature adjustable retention volumes, temperature adjustable premixing volumes and others known in the art.

It has been found that using a so-called microreactor is particularly advantageous for H-DIBAL reductions if used with multiple-injection points. According to the present method, improved control over a fluid H-DIBAL reduction can be achieved, which can result in significant improvements in reaction product yield and/or purity, as well as other benefits. The reaction starts after contacting the reactive fluids A and B in the mixing zone (3) and continues in a reaction zone (3). In a preferred embodiment the flow path(s) (1) has/have a width in the range of 10 to 10000 micrometers and a cross section of 0.1 square centimeters or less. More preferably the flow path width is in a range of 10 to 1000 micrometers, or even more preferably in a range of 10 to 500 micrometers. To prevent plugging of the microstructures with active ingredients a lower flow path width of about 200 micrometers is recommended.

In a further preferred embodiment heat or cooling independently is supplied to the reservoirs of reactants, injection point(s) (3), the mixing zone(s) (4) and/or the reaction zone(s) (5) or any other structural entity of the microreactor used. Preferably the heat or cooling is supplied by an external source. Said heat or cooling can be supplied to initiate, maintain and/or slow down the reaction. Preferably heat is supplied to initiate and/or maintain the reaction, whereas cooling is supplied to slow down the reaction. In rare cases heat may be supplied to slow down the reaction, whereas cooling may be supplied to initiate and/or maintain the reaction.

In case of fast reactions which essentially take place in the mixing zone the reaction zone can be used to adjust the temperature of the reaction mixture before injecting the next fraction of H-DIBAL to the compound to be reduced which is already present in the first flow.

Fast exothermic reactions which are almost completed when the reaction mixture has passed the mixing zone may require additional cooling while passing the reaction zone to suppress side product formation. Performing slow reactions often leads to side products when the reaction is forced to complete conversion. Generally, while performing H-DIBAL reactions the first flow (1) of fluids containing the reaction product is quenched after being discharged from the microreactor to prevent side reactions and to remove excess of H-DIBAL prior to work-up. In a preferred embodiment the product is isolated after quenching the reaction. In case the reaction does not reach completion in the mixing zone for several H-DIBAL reductions it may be suitable to accommodate the discharged first flow from the reaction zone or the microreactor into an external retention volume for further reaction before quenching, for other H-DIBAL reductions it may be suitable after the last injection point to quench the first flow directly after being discharged from the reaction zone or from the microreactor before it reaches completion to avoid over-reaction.

We have shown in the examples below that in H-DIBAL reductions the yield increases with the number of injection points. Comparing the benefit of each additional injection zone with the efforts and drawbacks connecting or building a further injection zone (new microreactor design, increase of hardware investment, additional programming work, increased fluid pressure, increased danger of leakage) it has been found, that the inventive method is advantageously carried out with a microreactor comprising not more than 7 reaction regions (injection points, mixing zones, reaction zones), preferably 3 to 6 reaction regions.

### DESCRIPTION OF THE DRAWINGS

FIG. 1 is a schematic drawing of a microreactor (6) comprising a flow path (1) through the whole microreactor and embedded three reaction regions (2) each, reaction region comprising an injection point (3), a mixing zone (4) and a reaction zone (5), wherein a fluid B is fed to a fluid A.

FIG. 2 shows a schematic drawing of a microreactor comprising two such flow paths.

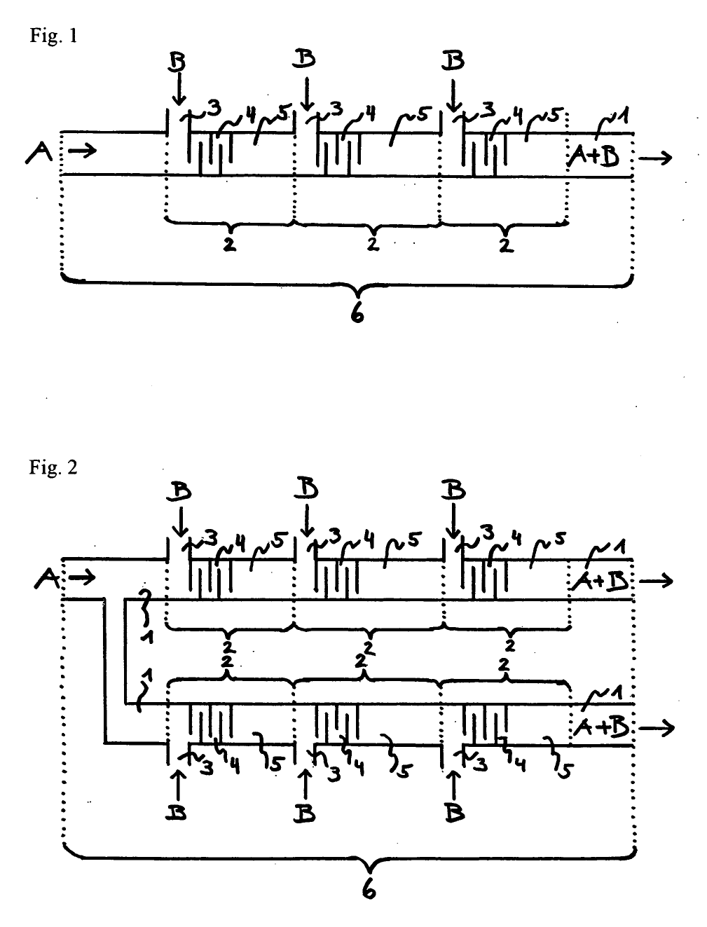

The invention is explained by the following examples which merely do not restrict the general inventive idea.

### EXAMPLES

The microreactors used in the examples and comparison example are made of different materials (glass or metal) and differently built systems. Some are integrated microreactor entities wherein injection point(s), mixing zone(s) and reaction zone(s) are built in one physical entity. Others are made from single elements (injection point(s), mixing zone(s) and reaction zone(s)) which are connected via external fittings. Some microreactors are temperature adjusted by immersing in a temperature controlled bath without having any additional elaborated temperature adjustment systems in place. Others also contain an efficient internal temperature adjustment system wherein a temperature controlled fluid is accommodated to the outside surface of injection point(s), mixing zone(s) and reaction zone(s) to provide an efficient and quick temperature adjustment. To facilitate the evaluation of the influence of the number of injection points in all examples H-DIBAL (2^{nd} reactant) is fed to a 1^{st} reactant in a proportional way corresponding to the number of inlet points. With two, three, four inlet points about 50, 33.3 or 25 mol-% of the 2^{nd} reactant necessary to reach completion of the reaction respectively are fed at each inlet point.

In all examples Feed-1 is related to the amount of the 1^{st} reactant in a fluid, while Feed-2 is related to the 2^{nd} reactant (H-DIBAL) in a fluid. In each example the amount of the respective reactant in the fluid is given in wt.-%. The results of examples 1 to 5 are given in Table 1, the results of Comparison Example 1 in Table 2. In the tables C, Y and S denote Conversion, Yield and Selectivity, respectively. In Examples 1 to 5 and Reference Example 1 methyl 1-butyrate has been reacted with H-DIBAL to give 1-butyraldehyde (=Aldehyde). The main side product is 1-butanol (Alcohol). With one exception in example 2.2 the feed of the 1^{st} reactant and the 2^{nd} reactant has been controlled to reach a molar Feed-2/Feed-1 ratio of 1.01 or above. Most examples have been reproduced several times with comparative results.

### Example 1

1^{st} reactant in Feed-1 has been set to 2.3 wt.-% and 2^{nd} reactant in Feed-2 has been set to 24.9 wt.-%. The reaction temperature has been set to 0° C.

### Example 2

1^{st} reactant in Feed-1 has been set to 10.0 wt.-% and 2^{nd} reactant in Feed-1 has been set to 24.9 wt.-%. The reaction temperature has been set to 0° C.

### Examples 3 to 5

1^{st} reactant in Feed-1 has been set to 2.3 wt.-% and 2^{nd} reactant in Feed-2 has been set to 24.9 wt.-%. The reaction temperature has been set to −20° C. In Examples 3 to 5 different pump types have been used to provide Feed-1 and Feed-2.

TABLE 1

| | | | | Total | | | | | |
|---|---|---|---|---|---|---|---|---|---|
| | # of inj. | Time | Feed-2/Feed-1 | flow | Aldehyde | Alcohol | C | Y | S |
| Exp. | points | [s] | ratio | [g/min] | [area %] | [area %] | [%] | [%] | [%] |
| 1.1 | 1 | 53 | 1.02 | 11.2 | 6.7 | 5.5 | 48.3 | 26.5 | 55.0 |
| 1.2 | 1 | 35 | 1.01 | 16.9 | 11.4 | 4.8 | 55.6 | 39.1 | 70.2 |
| 1.3 | 1 | 26 | 1.01 | 22.5 | 13.1 | 4.7 | 57.9 | 42.6 | 73.6 |
| 2.1 | 1 | 23 | 1.01 | 26.0 | 6.8 | 10.2 | 44.0 | 17.5 | 39.8 |
| 2.2 | 2 | 23 | 0.99 | 26.2 | 8.1 | 9.3 | 46.9 | 21.8 | 46.5 |
| 2.3 | 3 | 23 | 1.01 | 25.9 | 8.6 | 8.8 | 47.9 | 23.7 | 49.5 |
| 2.4 | 4 | 23 | 1.01 | 25.9 | 8.8 | 8.9 | 48.5 | 24.1 | 49.7 |
| 2.5 | 4 | 17 | 1.01 | 34.6 | 9.9 | 10.4 | 48.3 | 23.7 | 49.0 |
| 2.6 | 4 | 14 | 1.01 | 43.1 | 9.0 | 9.7 | 47.1 | 22.7 | 48.1 |
| 3.1 | 4 | 13 | 1.01 | 22.4 | 17.3 | 2.1 | 66.1 | 58.9 | 89.1 |
| 3.2 | 4 | 13 | 1.06 | 21.6 | 19.4 | 1.9 | 66.9 | 60.8 | 90.9 |
| 3.3 | 4 | 13 | 1.01 | 22.4 | 17.3 | 1.7 | 64.8 | 58.9 | 90.9 |
| 3.4 | 4 | 13 | 1.01 | 22.4 | 19.0 | 1.5 | 66.0 | 61.1 | 92.6 |
| 3.5 | 4 | 13 | 1.01 | 22.4 | 19.4 | 1.5 | 66.1 | 61.3 | 92.7 |
| 3.6 | 4 | 13 | 1.01 | 22.4 | 19.1 | 2.0 | 69.7 | 62.9 | 90.4 |
| 3.7 | 4 | 13 | 1.01 | 22.4 | 18.5 | 2.3 | 69.6 | 61.8 | 88.8 |
| 4.1 | 4 | 13 | 1.06 | 22.3 | 15.6 | 2.7 | 51.9 | 44.3 | 85.4 |
| 4.2 | 4 | 13 | 1.01 | 22.1 | 11.0 | 4.5 | 42.9 | 30.4 | 70.8 |
| 4.3 | 4 | 13 | 1.01 | 22.2 | 15.0 | 3.6 | 58.2 | 46.9 | 80.6 |
| 4.4 | 4 | 13 | 1.01 | 22.4 | 15.6 | 1.8 | 38.1 | 34.2 | 89.7 |
| 4.5 | 4 | 13 | 1.01 | 21.4 | 13.8 | 4.2 | 54.7 | 42.0 | 76.8 |
| 5.1 | 4 | 13 | 1.06 | 22.5 | 12.2 | 4.2 | 49.1 | 36.5 | 74.3 |
| 5.2 | 4 | 13 | 1.01 | 22.4 | 12.4 | 4.2 | 51.7 | 38.7 | 74.9 |
| 5.3 | 4 | 13 | 1.01 | 22.5 | 13.2 | 4.5 | 53.7 | 40.0 | 74.5 |
| 5.4 | 4 | 13 | 1.01 | 22.4 | 12.9 | 4.6 | 53.6 | 39.5 | 73.7 |
| 5.5 | 4 | 13 | 1.01 | 22.5 | 12.8 | 4.6 | 53.9 | 39.6 | 73.4 |

### Comparison Example 1

Feed-1 has been set to 2.3 wt.-% and Feed-2 has been set to 24.9 wt.-%. The reaction temperature has been set to 20° C.

TABLE 2

| | | | | Total | | | | | |
|---|---|---|---|---|---|---|---|---|---|
| C.- | # of inj. | Time | Feed-2/Feed-1 | flow | Aldehyde | Alcohol | C | Y | S |
| Exp. | points | [s] | ratio | [g/min] | [area %] | [area %] | [%] | [%] | [%] |
| 1.1 | 1 | 53 | 1.01 | 11.3 | 4.4 | 8.5 | 38.1 | 13.0 | 34.2 |
| 1.2 | 1 | 35 | 1.01 | 16.8 | 5.1 | 7.2 | 40.7 | 16.7 | 41.2 |
| 1.3 | 1 | 26 | 1.01 | 22.4 | 6.2 | 6.7 | 43.6 | 20.9 | 47.9 |

1. A method for the preparation of an aldehyde by reduction of an alkyl ester of a carboxylic acid, said acid being selected from the group consisting of aliphatic, alicyclic, aromatic, heterocyclic and heteroaromatic carboxylic acids, with H-DIBAL (diisobutyl-aluminium hydride), wherein the ester/H-DIBAL molar ratio is between about 1:1 and about 1:1.2, and wherein the reaction is carried out in a microreactor, and wherein the temperature in the microreactor is controlled in the range from 0 to −50° C., preferably in the range of −18 to −40° C., more preferably in the range of −20 to −40° C.

2. The method of claim 1, wherein the alkyl ester is a mixed ester, having at least one w-alkyl ester group and at least one branched alkyl ester group in the same molecule, and wherein the temperature of the microreactor is controlled to −18° C. or lower, preferably to −20° C. or lower, and wherein predominantly the w-alkyl ester group is reduced and the corresponding aldehyde group is obtained while the branched alkyl ester group remains essentially unaffected.

3. The method of claim 1, wherein the alkyl ester is selected from the group consisting of alkyl esters of acids selected of C₃₋₃o-aliphatic, alicyclic, aromatic, heterocyclic and heteroaromatic carboxylic acids.

4. The method of claim 1, wherein the n-alkyl ester is selected from the group consisting of methyl, ethyl, o-propyl and rc-butyl esters.

5. The method of claim 1, wherein the reaction time is below 1 min, preferably below 30 seconds.

6. The method of claim 1, comprising mixing at least two fluids, one of the at least two fluids comprising the ester of an aliphatic, aromatic and heteroaromatic acid (1st reactant), and another fluid comprising H-DIBAL (diisobutylaluminium hydride) (2nd reactant), and optionally further fluids, said mixing taking place in a microreactor comprising at least one flow path for one of the at least two fluids comprising either the 1st or 2nd reactant, said flow path(s) comprising at least two reaction regions, each reaction region comprising an injection point for feeding the other one of the two fluids comprising either the 2^{nd} or 1^{st} reactant, a mixing zone in which the at least two fluids contact each other and a reaction zone, and wherein the microreactor optionally provides one or more additional residence time volumes, and wherein in said method one of the at least two fluids comprising either the 1^{st} or 2^{nd} reactant establishes a first flow and wherein at other one of the at least two fluids comprising either the 2^{nd} or 1^{st} reactant is injected into said first flow at least at two injection points along said flow path(s) in a way such that at each injection point only a fraction of the amount necessary to reach completion of the reaction is injected.

7. The method of claim 6, wherein the flow path(s) (1) has/have a width in the range of 10 to 10000 micrometers and a cross section of 0.1 square centimeters or less.

8. The method of claim 6, wherein the flow path width is in a range of 10-1000 micrometers.

9. The method of claim 8, wherein the flow path width is in a range of 10 to 500 micrometers.

10. The method of claim 6, wherein heat or cooling independently is supplied to the injection point(s) (3), the mixing zone(s) (4) and/or the reaction zone(s) (5).

11. The method of claim 6, wherein heat or cooling is supplied to initiate, maintain and/or slow down the reaction.

12. The method of claim 6, wherein heat is supplied to initiate and/or maintain the reaction.

13. The method of claim 6, wherein cooling is supplied to slow down the reaction.

14. The method of claim 6, wherein the microreactor (6) comprises 3 to 6 reaction regions (2).

15. The method of claim 6, wherein in slow reactions the reaction is quenched after the last reaction zone before it reaches completion.
